(19) United States
(12) Patent Application Publication
(10) Pub. No.: US 2017/0020766 A1
(43) Pub. Date: Jan. 26, 2017

(54) METHOD OF RESTORING BINOCULAR FUSION AND STEREOPSIS

(71) Applicant: Igor Aznauryan, Moscow (RU)

(72) Inventor: Igor Aznauryan, Moscow (RU)

(21) Appl. No.: 14/809,016

(22) Filed: Jul. 24, 2015

(51) Int. Cl. A61H 5/00; G02F 1/13; A61B 3/08

(52) U.S. Cl. CPC A61H 5/00; A61B 3/085; G02F 1/1313

(57) ABSTRACT

A method of restoring binocular fusion and stereopsis in a patient exhibiting a functional scotoma includes determining that the patient has a treatable scotoma, presenting an object to be viewed by the patient, placing an eyewear apparatus with a right lens and a left lens in front of the patient's eyes, and treating the scotoma by alternately and sequentially occluding the right lens and the left lens at a frequency of 0.5-10 Hz when the patient's gaze is fixed on the object.

### BACKGROUND OF THE INVENTION

The present invention relates to a method for restoring binocular fusion and stereopsis in patients with functional scotomas caused by strabismus that is congenital or acquired early in life and persisting for a long time.

Patients, especially children, with strabismus can develop a binocular fusion and stereopsis disorder which causes the patient's brain to eliminate or suppress the symptoms by ignoring all or part of one of the eyes. The suppressed portion is referred to as a scotoma. The binocular fusion and stereopsis disorder caused thereby is characterized by the patient's inability to merge two monocular images in one combined visual image.

After a surgery to correct the strabismus, a majority of patients maintain the scotoma. Currently, there is no treatment for the scotoma binocular fusion and stereopsis disorder.

### BRIEF SUMMARY OF THE INVENTION

An object of the present invention is to provide a method for restoring binocular fusion and stereopsis in patients with scotomas.

According to an embodiment of the invention, visual perceptions on both eyes are changed by alternating dark/clear cycles in patients with orthotropia to stimulate alleviation of the suppression of binocular system and restore binocular fusion and stereopsis. In the present application and claims, orthotropia is considered to be a residual strabismus of up to ±15 degrees. More specifically, the method according to the present invention is effective to restore binocular fusion and stereopsis, i.e., eliminate the scotoma, in patients exhibiting a residual strabismus of not more than ±15 degrees.

According to an embodiment of the invention, a method of restoring binocular fusion and stereopsis in a patient exhibiting a functional scotoma includes determining that the patient has a treatable scotoma, presenting an object to be viewed by the patient, placing an eyewear apparatus with a right lens and a left lens in front of the patient's eyes, and alternately and sequentially occluding the right lens and the left lens when the patient's gaze is fixed on the object.

The right lens and the left lens each include a liquid crystal shutter.

The step of alternately and sequentially occluding includes opening and shutting each of the right lens and the left lens at a frequency of occlusion between 0.5 to 10 Hz. The frequency of occlusion to be used based on a residual strabismus angle exhibited by the patient. The frequency of occlusion is set to 0.5 Hz when the residual strabismus angle is approximately ±15 degrees and is set to 10 Hz when the residual strabismus angle is approximately 0 degrees.

The step of setting is performed using a user interface on the eyewear apparatus or by connecting the eyewear apparatus to a computer and using the computer as an interface.

The step of alternately and sequentially occluding is performed continuously for 30 to 120 minutes per day, for 10-15 days.

The step of alternately and sequentially occluding includes opening and closing the shutter for equal amounts of time. Alternatively, the time of closing the shutter is longer or shorter than the time of opening the shutter.

According to an embodiment, the distance between the lenses is adjusted based on the inter-pupillary distance of the patient.

The present invention also relates to eyewear apparatus for treating a scotoma in a patient, including a frame having two lenses corresponding to the patients two eyes, two LC cells respectively arranged in the two lenses, a circuit configured to alternately and sequentially occlude the two lenses by actuating the LC cells.

In one embodiment, a user interface is connected to the circuit, and the circuit is configured to set a frequency of occluding the two holes based on a user input entered on the user interface.

In another embodiment, the eyewear apparatus is connectable to a computer, and the circuit is configured to set a frequency of occluding the two holes based on a user input entered on the computer.

### BRIEF DESCRIPTION OF THE DRAWINGS

In the drawings:

FIG. 1 is a flow diagram of the method according to an embodiment of the present invention;

FIG. 2 is a schematic perspective view of an eyewear apparatus according to the present invention;

FIG. 3 is a schematic cross-sectional perspective view of an eyewear apparatus according to another embodiment of the present invention;

FIGS. 4A, 4B, and 4C are each a schematic view of a part of the eyewear apparatus according to different embodiments of the present invention;

FIG. 5 is a graph showing open and closing times of the eyeglass shutters according to an embodiment of the present invention;

FIGS. 6A and 6B are graphs showing open and closing times of the eyeglass shutters according to different embodiments of the present invention;

FIG. 7 is a graph showing frequency settings based on residual angle of strabismus;

FIGS. 8A and 8B are top views of an embodiment of the eyewear apparatus according to the invention;

FIG. 9 is a bar graph showing the results of the treatment according to the invention compared to results with no treatment; and

FIG. 10 is a schematic perspective view of an eyewear apparatus according to another embodiment of the present invention.

### DETAILED DESCRIPTION OF THE PREFERRED EMBODIMENTS

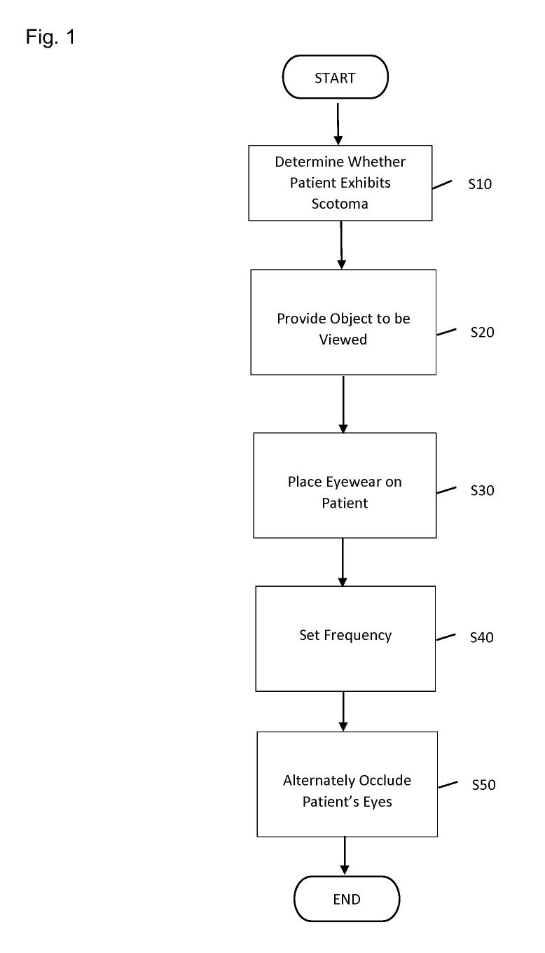

According to FIG. 1, a first step according to an embodiment of the present invention is to determine whether a patient exhibits a functional scotoma, step S10. A functional scotoma is a binocular fusion and stereopsis disorder in which the ability to merge images from the left eye and the right eye when the images are each projected onto a retina's fovea centralis is absent. In step S10, a synoptophore can be used by showing each eye a separate image with a certain respective object. The objects are presented to the patient so that they are projected onto corresponding areas of the fovea centralis (central part of the retina) of the respective eyes. If the patient does not have a functional scotoma, the objects will merge into one. However, if a functional scotoma is present, the images are not combined and the patient will see two separate images.

Once it is determined that a patient has the functional scotoma, a treatment is prepared. The treatment includes providing an object to be viewed such as, for example, a monitor with a movie or other program of interest to the patient, step S20, placing an eyewear apparatus with two lenses in front of the patient's eyes so that the patient views the object through the two lenses in the eyewear apparatus, step S30, setting a frequency of occlusion, step S40, and alternately shuttering the eyes with a frequency of 0.5-10 Hz, step S50.

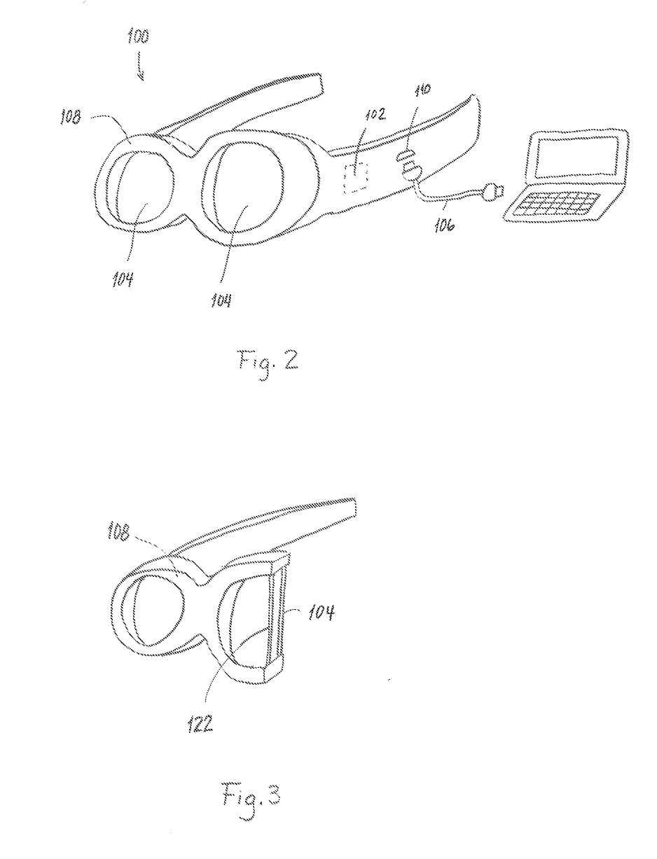

FIG. 2 shows a schematic view of an eyewear apparatus 100 according to an embodiment of the present invention. The eyewear apparatus 100 includes a frame 108 with two LC lenses 104, 104 connected a programmable controller circuit 102 that is embedded in the frame 108. The LC lenses include liquid crystal cells, which can be controlled by the programmable controller circuit 102 to be transparent in one state, i.e., when no power is supplied to the LC lens, and opaque or shuttered in another state, i.e., when power is supplied to the LC lens. In the FIG. 2 embodiment, the frame 108 also includes a port 110 for a USB connection 106 to a computer. Thus, the eyewear apparatus 100 can be connected to the computer via a USB connection similar to known computer peripheral devices. The frequency of occlusion and the percentage of time that the lens is closed during one cycle can be set using the computer. Instead of a USB connection, any other known wired or wireless connection to the computer may be used such as, for example, Bluetooth, wi-fi, or Ethernet connections.

According to one embodiment, the monitor on which the object to be viewed is displayed is separate from the glasses, i.e., a standalone display. In another embodiment shown in FIG. 3, the glasses may contain the monitor 122 or other known or hereafter developed display screen to display the object to be viewed. In the FIG. 3 embodiment, the LC lens 104 is arranged between the monitor 122 and the patient's eye when the glasses are worn.

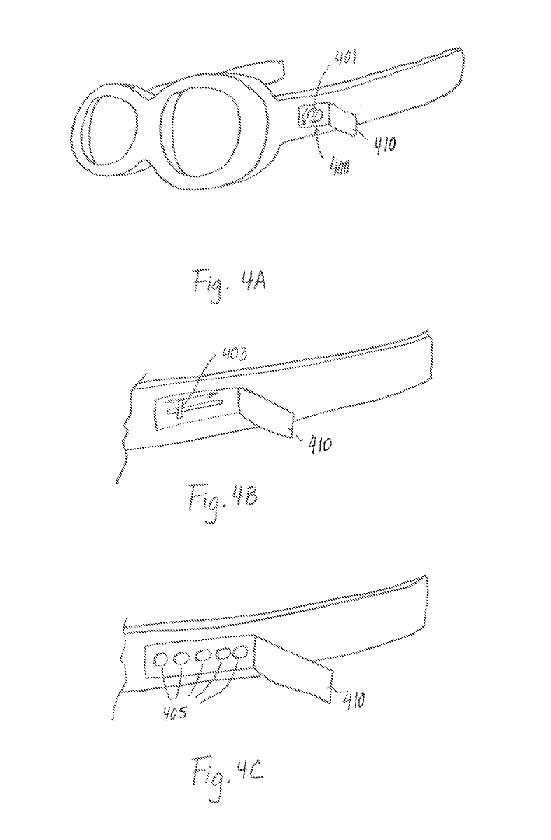

In further alternative embodiments shown in FIGS. 4A, 4B, and 4C, the glasses contain a user interface 400 to change the settings without requiring a connection to the computer. FIG. 4A shows a rotating dial 401 that can be used to set the frequency, FIG. 4B shows a sliding dial 403, and FIG. 4C shows separate buttons 405 for each setting. For example, in FIG. 4C, each of the buttons 405 could respectively correspond to 0.5 Hz, 2.9 Hz, 5.25 Hz, 7.6 Hz, and 10 Hz. Although five buttons are disclosed in FIG. 4C, any intervals and any number of buttons could be used. As yet a further alternative, a step up and step down button could be used to increase or decrease the setting by a specific increment such as, for example, 0.5 Hz. In each of the embodiments shown in FIGS. 4A, 4B, 4C, a cover 410 in the form of a hinged door is used to cover the user interface to prevent inadvertent adjustments after the setting is made. Instead of a door, any cover such as a flexible flap that can be peeled away and re-adhere to the glasses can be used.

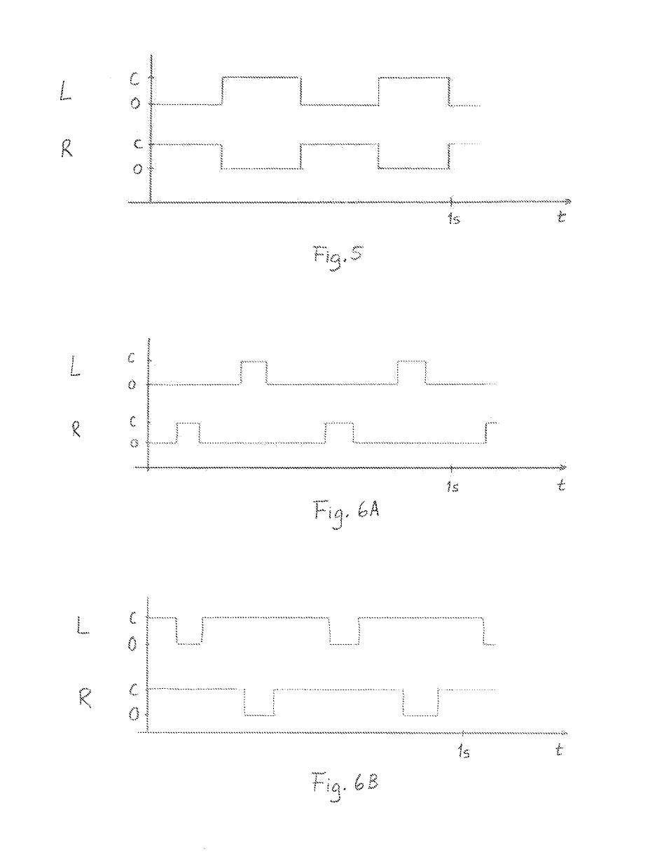

The frequency of occlusion indicates a total number of occlusions per second. That is, if the frequency is 4 Hz, each eye will be occluded twice every second as shown in FIG. 5. In that example, the left shutter L is open while the right shutter R is closed and vice versa, such that the occlusion time is equal to the open time for each hole. In another embodiment, the time that a lens is open may be different from the time that the lens is closed. As shown in FIG. 6A, the occlusion time is smaller than the open time. In yet another embodiment shown in FIG. 6B, the occlusion time is greater than the open time.

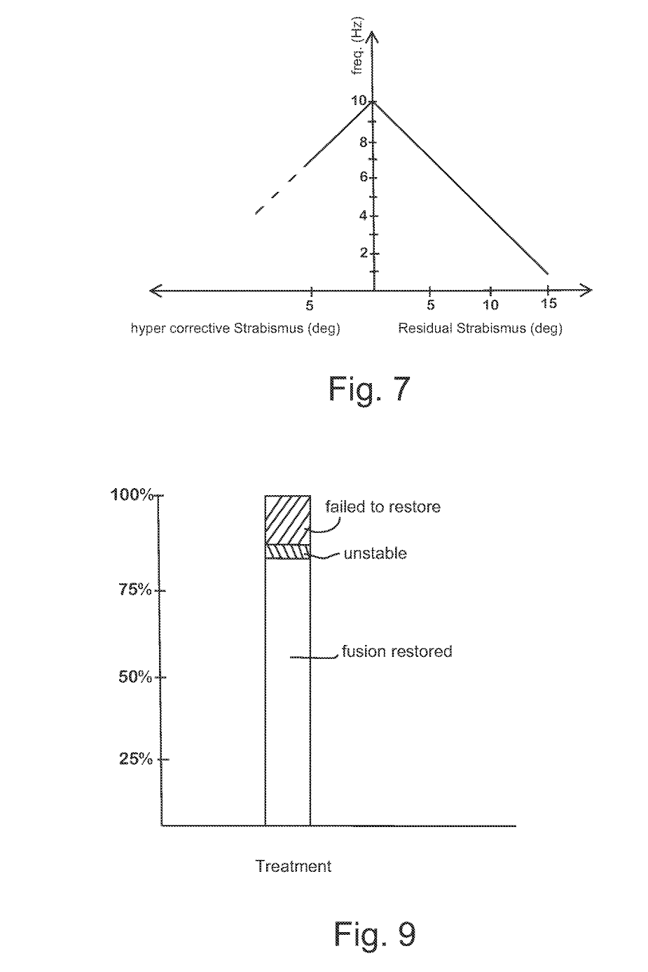

The frequency of the treatment depends on the angle of strabismus, i.e., residual strabismus after surgery. In other embodiments the frequency may alternatively or additionally depend on the strabismus before surgery. Lower frequencies are used when there is a larger angle of strabismus. The method of the present invention is found to be effective with residual angle of strabismus of ±15 degrees or less. As shown in FIG. 7, if the angle of residual strabismus is ±15 degrees, the frequency is set to the lowest setting, i.e., 0.5 Hz. The highest frequency setting of 10 Hz is used for an angle of residual strabismus of approximately 0 degrees. The frequency is interpolated between 0.5 Hz and 10 Hz for angles of strabismus between ±15 degrees and 0 degrees.

In some cases, the surgery for strabismus will result in a hyper correction or an over correction. The hypercorrection is usually less than ±5 degrees. This is depicted on the left side of the graph of FIG. 7. The same frequency that is used for the residual strabismus is used for the hyper-corrected strabismus.

Steps S20, S30, and S50 are performed daily on the patient for a period of 30-120 minutes for 10-15 days. The glasses can be designed so that the programmable circuit retains the last used frequency setting. Accordingly, the setting of the frequency, step S40, may not have to be performed for each daily treatment session after the initial setting.

The lenses in the eyewear apparatus are at a distance from each other that is determined by an inter-pupillary distance of the patients' eyes. This can be achieved by using various sizes of eyewear apparatus or alternatively adjusting a bridge 118 between the two holes, i.e, by bending, as shown in FIGS. 8A and 8B.

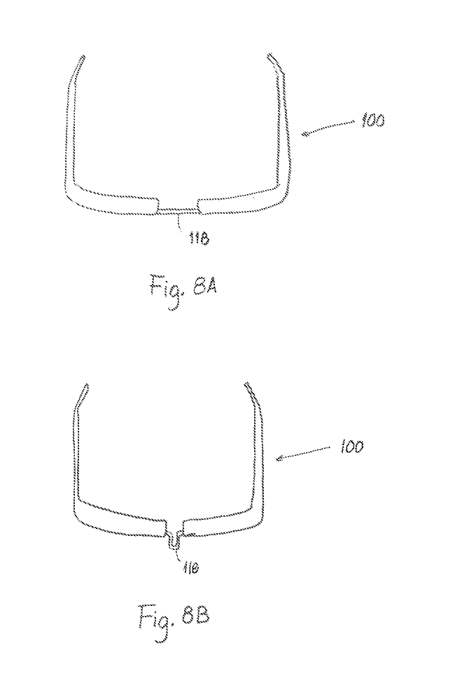

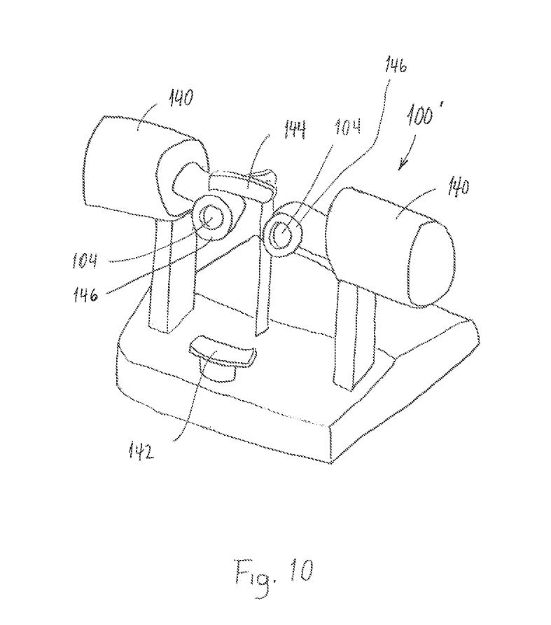

147 patients having binocular fusion and stereopsis disorder were treated using the above-described method aged 4-13. Visual acuity of the patients varied from 20/25 to 20/20. All of the patients were determined to have functional scotoma with a synoptophore according to step S10 described above. The patients exhibited angles of strabismus after surgery of ±4 to ±6 degrees. In 133 of the 147 patients, bifoveal fusion and stereopsis and binocular vision (orthophoria) was fully restored. In 5 of the 147 patients, the ability to fuse was unstable. In 9 of the 147 patients, the method failed to restore binocular fusion and stereopsis. Thus, as shown in FIG. 9, the treatment according to the invention resulted 85% of patients after a surgery to restore binocular fusion and stereopsis. In contrast, a prior study has shown that only a small number of patients exhibit improvement in binocular fusion and stereopsis after treatment of the strabismus, i.e., without the inventive treatment after correction of the strabismus. In this prior study, a majority of patients treated for strabismus retain a binocular fusion and stereopsis disorder. Levi, D. M., et al., Stereopsis and amblyopia: a mini-review, Vision Research (2015).

According to the invention, after the initial treatment is performed, patients are checked monthly for a period of 6 months to ensure that the binocular fusion and stereopsis disorder has not developed again or returned. If after six months, the binocular fusion and stereopsis disorder has not returned, i.e., binocular fusion and stereopsis is stable, there is no need to repeat the treatment. However, if during one the monthly check-ups, the binocular fusion and stereopsis is found to be impaired, the treatment shown in FIG. 1 and described above is repeated until binocular fusion and stereopsis is stable for 6 months. In the sample of 147 patients treated by the applicant described above, treatment was repeated 1-3 times in 25% of the cases. Applicant has found the probability that the treatment will have to be repeated increases with the patient's age at the time that the strabismus surgery was diagnosed and conducted. The older the patient and the later the surgery was conducted, the more likely the treatment is required to be repeated.

Although FIGS. 2 and 3 show an eyewear apparatus 100 that is worn by a user, in an alternative embodiment shown in FIG. 10, the eyewear apparatus 100′ can be a stationary device that requires the patient to be positioned at the device. The stationary device has eyepieces 146 and chin holder 142 and a forehead support 144. Housings 140 include controls for occluding the lenses 104 in the eyepieces. The user views an object through the eyepieces 146 while the occlusions occur. In a further embodiment, the eyewear apparatus 100′ show the object to be viewed on a monitor 122 as described above in addition to the occluding lenses.

Thus, while there have been shown and described and pointed out fundamental novel features of the invention as applied to a preferred embodiment thereof, it will be understood that various omissions and substitutions and changes in the form and details of the devices illustrated and described, and in their operation, may be made by those skilled in the art without departing from the scope of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention therefore, to be limited only by the scope of the claims appended hereto.

What is claimed is:

1. A method of restoring binocular fusion and stereopsis in a patient exhibiting a functional scotoma, comprising the steps of:
determining that the patient has a treatable scotoma;
presenting an object to be viewed by the patient;
placing an eyewear apparatus with a right lens and a left lens in front of the patient's eyes; and
treating the scotoma by alternately and sequentially occluding the right lens and the left lens at a frequency of 0.5-10 Hz when the patient's gaze is fixed on the object.

2. The method of claim 1, wherein the right lens and the left lens each comprise a liquid crystal shutter.

3. The method of claim 1, further comprising the step of adjusting the frequency of occlusion based on a residual strabismus angle exhibited by the patient.

4. The method of claim 3, wherein the frequency of occlusion is set to 0.5 Hz when the residual strabismus angle is approximately ±15 degrees and is set to 10 Hz when the residual strabismus angle is approximately 0 degrees and is interpolated for strabismus angles between 0 and ±15 degrees.

5. The method of claim 3, wherein the step of adjusting is performed using a user interface on the eyewear apparatus.

6. The method of claim 3, wherein the step of adjusting is performed by connecting the eyewear apparatus to a computer and using the computer as an interface.

7. The method of claim 1, wherein the step of alternately and sequentially occluding is performed continuously for 30 to 120 minutes per day.

8. The method of claim 7, wherein the method is performed daily for 10-15 days.

9. The method of claim 1, wherein the step of alternately and sequentially occluding includes opening and closing the lenses for equal amounts of time.

10. The method of claim 1, wherein the step of alternately and sequentially occluding is performed such that the time of closing each of the lenses is longer than the time of opening each of the lenses.

11. The method of claim 1, wherein the step of alternately and sequentially occluding is performed such that the time of opening each of the lenses is longer than the time of closing the each of the lenses.

12. The method of claim 1, further comprising the step of adjusting the distance between the holes based on the inter-pupillary distance of the patient.

13. The method of claim 1, wherein the treatable scotoma is a scotoma in a patient with a residual angle of strabismus that is not greater than ±15 degrees.

14. Eyewear apparatus for treating a scotoma in a patient, comprising:
a frame having two lenses corresponding to the patients two eyes;
two LC cells respectively arranged in the two lenses;
a circuit configured to alternately and sequentially occlude the two lenses at a frequency of 0.5-10 Hz by actuating the LC cells; and
one of a user interface connected to the circuit, wherein the circuit is configured to set a frequency of occluding the two lenses based on a user input entered on the user interface, and a port connectable to a computer, wherein the circuit is configured to set a frequency of occluding the two holes based on a user input entered on the computer.
